SEQUENCE LISTING (2 sequences)

SEQ ID NO: 1 - protein, 966 aa, Trichoderma sp.
  misc_feature: “TrTG” - mature transglucosidase enzyme
ASQSLLSTTAPSQPQFTIPASADVGAQLIANIDDPQAADAQSVCPGYKASKVQHNSRGFTASLQLAGRPCNVYGTDVESLTLSVEYQDSDRLNIQILPTHVDSTNASWYFLSENLVPRPKASLNASVSQSDLFVSWSNEPSFNFKVIRKATGDALFSTEGTVLVYENQFIEFVTALPEEYNLYGLGEHITQFRLQRNANLTIYPSDDGTPIDQNLYGQHPFYLDTRYYKGDRQNGSYIPVKSSEADASQDYISLSHGVFLRNSHGLEILLRSQKLIWRTLGGGIDLTFYSGPAPADVTRQYLTSTVGLPAMQQYNTLGFHQCRWGYNNWSDLADVVANFEKFEIPLEYIWTDIDYMHGYRNFDNDQHRFSYSEGDEFLSKLHESGRYYVPIVDAALYIPNPENASDAYATYDRGAADDVFLKNPDGSLYIGAVWPGYTVFPDWHHPKAVDFWANELVIWSKKVAFDGVWYDMSEVSSFCVGSCGTGNLTLNPAHPSFLLPGEPGDIIYDYPEAFNITNATEAASASAGASSQAAATATTTSTSVSYLRTTPTPGVRNVEHPPYVINHDQEGHDLSVHAVSPNATHVDGVEEYDVHGLYGHQGLNATYQGLLEVWSHKRRPFIIGRSTFAGSGKWAGHWGGDNYSKWWSMYYSISQALSFSLFDIPMFGADTCGFNGNSDEELCNRWMQLSAFFPFYRNHNELSTIPQEPYRWASVIEATKSAMRIRYAILPYFYTLFDLAHTTGSTVMRALSWEFPNDPTLAAVETQFMVGPAIMVVPVLEPLVNTVKGVFPGVGHGEVWYDWYTQAAVDAKPGVNTTISAPLGHIPVYVRGGNILPMQEPALTTREARQTPWALLAALGSNGTASGQLYLDDGESIYPNATLHVDFTASRSSLRSSAQGRWKERNPLANVTVLGVNKEPSAVTLNGQAVFPGSVTYNSTSQVLFVGGLQNLTKGGAWAENWVLEW

SEQ ID NO: 2 - DNA, 3046 bp, Trichoderma sp.
  misc_feature: “TrTG” - mature transglucosidase enzyme
tccaccactgccccttcgcagccgcagtttaccattcctgcttccgcagatgtcggtgcgcagctgattgccaacatcgatgatcctcaggctgccgacgcgcagtcggtttgtccgggctacaaggcttcaaaagtgcagcacaattcacgtggattcactgccagtcttcagctcgcgggcaggccatgtaacgtatacggcacagatgttgagtccttgacactgtctgtggagtaccaggattcggatcgactgaatattcagattctccccactcatgttgactccacaaacgcttcttggtactttctttcggaaaacctggtccccagacccaaggcttccctcaatgcatctgtatcccagagcgacctttttgtgtcatggtcaaatgagccgtcgttcaatttcaaggtgatccgaaaggctacaggcgacgcgcttttcagtacagaaggcactgtgctcgtatatgagaatcagttcatcgaatttgtgaccgcgctccctgaagaatataacttgtatggccttggggagcatatcacgcaattccgcctccagagaaatgctaatctgaccatatatccttcggatgatggaacacctattgaccagtgagtactgatatcccgcccgtatcttctggttctactcttgaaacttactcgtcctagaaacctctacggccaacatcccttctatctggatacaagatattacaaaggagataggcagaatgggtcttatattcccgtcaaaagcagcgaggctgatgcctcgcaagattatatctccctctctcatggcgtgtttctgaggaactctcatggacttgagatactcctccggtctcaaaaattgatctggcggaccctaggtggaggaatcgatctcaccttctactcaggccccgccccggccgatgttaccaggcaatatcttaccagcactgtgggattaccggccatgcagcaatacaacactcttggattccaccaatgtcgttggggctacaacaactggtcggatctggcggacgttgttgcgaactttgagaagtttgagatcccgttggaatatatctggtgcgtattgtactggtttatggtatctcaaaacagtctaacaggcacttaggaccgatattgactacatgcacggatatcgcaactttgacaacgatcaacatcgcttttcctacagtgagggcgatgaatttctcagcaagctacatgagagtggacgctactatgtacccattgttgatgcggcgctctacattcctaatcccgaaaatgcctctgatgcgtaagtgtctagtgacaaattatattactgcctgtatgctaattagcgatacagatacgctacgtatgacagaggagctgcggacgacgtcttcctcaagaatcccgatggtagcctctatattggagccgtttggccaggatatacagtcttccccgattggcatcatcccaaggcagttgacttctgggctaacgagcttgttatctggtcgaagaaagtggcgttcgatggtgtgtggtacgacatgtctgaagtttcatccttctgtgtcgggagctgtggcacaggtaacctgactctgaacccggcacacccatcgtttcttctccccggtgagcctggtgatatcatatatgattacccagaggctttcaatatcaccaacgctacagaggcggcgtcagcttcggcgggagcttccagtcaggctgcagcaaccgcgaccaccacgtcgacttcggtatcatatctgcggacaacgcccacgcctggtgtccgcaatgttgagcacccaccctatgtgatcaaccatgaccaagaaggccatgatctcagtgtccatgcggtgtcgccgaatgcaacgcatgttgatggtgttgaggagtatgatgtgcacggtctctacggacatcaaggattgaacgctacctaccaaggtctgcttgaggtctggtctcataagcggcggccatttattattggccgctcaaccttcgctggctctggcaaatgggcaggccactggggcggcgacaactattccaaatggtggtccatgtactactccatctcgcaagccctctccttctcacttttcggcattccgatgtttggtgcggacacctgtgggtttaacggaaactccgatgaggagctctgcaaccgatggatgcaactgtccgcattcttcccattctaccgaaaccacaatgagctctccacaatcccacaggagccttatcggtgggcttctgttattgaagcaaccaagtccgccatgagaattcggtacgccatcctaccttacttttatacgttgtttgacctggcccacaccacgggctccactgtaatgcgcgcactttcctgggaattccctaatgacccaacattggctgcggttgagactcaattcatggttgggccggccatcatggtggtcccggtattggagcctctggtcaatacggtcaagggcgtattcccaggagttggacatggcgaagtgtggtacgattggtacacccaggctgcagttgatgcgaagcccggggtcaacacgaccatttcggcaccattgggccacatcccagtttatgtacgaggtggaaacatcttgccgatgcaagagccggcattgaccactcgtgaagcccggcaaaccccgtgggctttgctagctgcactaggaagcaatggaaccgcgtcggggcagctctatctcgatgatggagagagcatctaccccaatgccaccctccatgtggacttcacggcatcgcggtcaagcctgcgctcgtcggctcaaggaagatggaaagagaggaacccgcttgctaatgtgacggtgctcggagtgaacaaggagccctctgcggtgaccctgaatggacaggccgtatttcccgggtctgtcacgtacaattctacgtcccaggttctctttgttggggggctgcaaaacttgacgaagggcggcgcatgggcggaaaactgggtattggaatgg

(19) United States
(12) Patent Application Publication
(10) Pub. No.: US 2016/0257977 A1
(43) Pub. Date: Sep. 8, 2016

(54) Enhanced Fermentation Process Using a Transglycosidase

(71) Applicant: Danisco US Inc., Palo Alto, CA (US)

(72) Inventors: Gang Duan, Shanghai (CN); Jayarama K. Shetty, Pleasanton, CA (US); Xin Wang, Wuxi (CN); Hongxian Xu, Wuxi (CN); Xiaoping Zhang, Wuxi (CN); Peng Zhou, Wuxi (CN)

(73) Assignee: Danisco US Inc., Palo Alto, CA (US)

(21) Appl. No.: 15/031,459

(22) PCT Filed: Oct. 21, 2014
(86) PCT No.: PCT/US2014/061529

§ 371 Date: Apr. 22, 2016

(30) Foreign Application Priority Data
Oct. 24, 2013 (CN) PCT/CN2013/085866

(51) Int. Cl. C12P 13/14; C12P 7/56; C12P 13/08

(52) U.S. Cl. CPC C12P 13/14; C12P 13/08; C12P 7/56

(57) ABSTRACT

The present invention relates to methods for improving fermentation processes, including increasing product yield, reduced viscosity, and/or reduced foaming.

### CROSS REFERENCE TO RELATED APPLICATIONS

This application claims benefit of priority from International Patent Application No. PCT/CN2013/085866 filed on 24 Oct. 2013, and the contents of which are incorporated herein by reference in entirety.

### FIELD OF THE INVENTION

The present invention relates to the use of a transglycosidase enzyme preparation to improve fermentation processes. Methods of screening for suitable fermentation hosts and fermentation products amenable to improvement are also provided.

### DESCRIPTION OF THE RELATED ART

Industrial biotechnology represents the third wave in biotechnology and a growing number of companies are now focusing on specialty chemicals as an entry point to build the bio-based economy. Glutamic acid, lysine, lactic acid are top targets for further research and development within industrial biotechnology. The objective of a bio-refinery is to develop as more product and value streams as possible from biomass. Background summaries of the current technologies of bio-refinery fermentation can be found in Eggeling, L. and M. Bott (2010). Handbook of Corynebacterium glutamicum, CRC press; Zahoor, A., S. N. Lindner, et al. (2012). “Metabolic engineering of Corynebacterium glutamicum aimed at alternative carbon sources and new products.” Computational and Structural Biotechnology Journal 3(4); Ikeda, M. and S. Takeno (2013). “Amino Acid Production by Corynebacterium glutamicum.” 23: 107-147.

Current challenges and objectives include maximizing product yield from raw materials. While much research has been carried out in this area, including strain modification, various raw materials utilization, and process optimization, yields remain far from theoretical maximums. Another current problem is managing the foaming level of aerobic fermentation. Under certain conditions, the existence of the foam phase in bioreactors may be regarded as a positive factor enhancing mass transfer and growth of the given culture. On the other hand, intensive foam formation frequently reduces the actual volume of the liquid. Furthermore, foam ejection and loss of the fermentation product may occur, which negatively impacts manufacturing plant capacity. Also, contamination occurs as a consequence of overflowing foam, so antifoam is often required to control the foam, which can be quite costly and negatively impact the downstream processing used to recover the end-products. In aerobic fermentation, often the maximum amount of fermentation broth per reactor is governed by foaming, thus filling degree (determined as mass per reactor volume) varies between 70 and 85% in industrial practices. Foaming is influenced by the density of the fermentation broth, surface composition of the production strain, media composition, process conditions, viscosity of the fermentation medium, and so on. Though there are some studies focusing on foam formation (see e.g. Eggeling, L., K. Krumbach, et al. (2001). “L-glutamate efflux with Corynebacterium glutamicum: why is penicillin treatment or Tween addition doing the same?” Journal of molecular microbiology and biotechnology 3(1): 67-68; Eggeling, L. and H. Sahm (2001). “The cell wall barrier of Corynebacterium glutamicum and amino acid efflux.” Journal of bioscience and bioengineering 92(3): 201-213; Eggeling, L. and M. Bott (2010). Handbook of Corynebacterium glutamicum, CRC press; Yang, Y., F. Shi, et al. (2012). “Purification and structure analysis of mycolic acids in Corynebacterium glutamicum.” The Journal of Microbiology 50(2): 235-240), there no reports on foaming reduction by using additional enzymes. Another area of current research is directed to improving downstream process efficiency of bio-refinery fermentations. Recovery processes of amino acids and organic acids from fermentation broth may include conventional chromatographic method, concentrated crystallization method, direct drying method, calcium salt precipitation, and so on. Centrifugation, filtration, ultra-filtration, crystallization, spray-drying, etc are also often used to recover fermentation products.

### SUMMARY OF THE INVENTION

A method of making a fermentation product comprising; fermenting a feedstock with a fermenting microorganism in a fermentation broth to make a fermentation product, wherein the fermenting comprises a transglycosidase, and wherein the fermentation product has a higher yield than a control fermentation lacking the transglycosidase. In some embodiments, the method further comprises recovering the fermentation product. In some embodiments, the fermentation product is lysine, lactic acid, or glutamic acid. In some embodiments, the fermenting organism is Brevibacterium flavum, Corynebacterium glutamicum 10178, Corynebacterium glutamicum 10238, or Lactobacillus rhamnosus. In some embodiments, the transglycosidase comprises SEQ ID NO:1, or anything 80%, 85%, 90%, 95%, 98%, 99% identical to it. In some embodiments, the transglycosidase is produced in a Trichoderma. In some embodiments, the feedstock comprises starch liquifact, granular starch, pure glucose, pure sucrose, or sugar syrup. In some embodiments, the method further comprises foam reduction and/or viscosity reduction in the fermentation broth. In some embodiments, the Transglucosidase enzyme preparation (TrTG) is present at 1-6 Kg/MT, 2-5 Kg/MT, or 2-4 Kg/MT dry solids of fermentation feedstock.

In some embodiments, the present teachings provide a method of screening for a fermentation benefit to identify a beneficial transglycosidase comprising; fermenting a feedstock with a fermenting microorganism in a fermentation broth to make a fermentation product, wherein the fermenting comprises a transglycosidase, and wherein the fermentation product has a higher yield than a control fermentation lacking the transglycosidase; and, identifying the beneficial transglycosidase therefrom. In some embodiments, the fermentation product is lysine, lactic acid, or glutamic acid.

In some embodiments, the present teachings provide a method of screening for a fermentation benefit to identify a beneficial transglycosidase comprising; fermenting a feedstock with a fermenting microorganism in a fermentation broth to make a fermentation product, wherein the fermenting comprises a transglycosidase, and wherein the fermentation comprising foam reduction and/or viscosity reduction in the fermentation broth as compared to a control reaction lacking the transglycosidase; and, identifying the beneficial transglycosidase therefrom.

In some embodiments, the present teachings provide a method of making lysine comprising; fermenting a feedstock with Brevibacterium flavum in a fermentation broth to make lysine, wherein the fermenting comprises a TrTG, wherein the lysine has a higher yield compared to a control fermentation lacking the TrTG, and wherein the fermentation broth has reduced viscosity compared to a control fermentation lacking the TrTG.

In some embodiments, the present teachings provide a method of making lysine comprising; fermenting a feedstock with Brevibacterium flavum in a fermentation broth to make lysine, wherein the fermenting comprises an A. Niger TG, wherein the lysine has a higher yield compared to a control fermentation lacking the A. Niger TG.

In some embodiments, the present teachings provide a method of making lysine comprising; fermenting a feedstock with Brevibacterium flavum in a fermentation broth to make lysine, wherein the fermenting comprises a Transglucosidase, wherein the lysine has a higher yield compared to a control fermentation lacking the Transglucosidase. In some embodiments, the feedstock comprises pure glucose or sugar syrup.

In some embodiments, the present teachings provide a method of making lysine comprising; fermenting a feedstock with Brevibacterium flavum in a fermentation broth to make lysine, wherein the fermenting comprises a betaglucanase, xylanase and cellulase enzyme complex, wherein the lysine has a higher yield compared to a control fermentation lacking the betaglucanase, xylanase and cellulase enzyme complex.

In some embodiments, the present teachings provide a method of making glutamic acid comprising; fermenting a feedstock with Corynebacterium glutamicum SP 10238 in a fermentation broth to make glutamic acid, wherein the fermenting comprises a TrTG, wherein the fermentation broth has reduced viscosity compared to a control fermentation lacking the TrTG, and wherein the fermentation broth has reduced foam compared to a control fermentation lacking the TrTG.

In some embodiments, the present teachings provide a method of making glutamic acid comprising; fermenting a feedstock with Corynebacterium glutamicum SP 10238 in a fermentation broth to make glutamic acid, wherein the fermenting comprises an exoglucanase, a endoglucanase, a β-glucosidase, and a xylanase, wherein the fermentation broth has reduced viscosity compared to a control fermentation lacking the exoglucanase, the endoglucanase, the β-glucosidase, and the xylanase, and wherein the fermentation broth has reduced viscosity compared to a control fermentation lacking the exoglucanase, the endoglucanase, the β-glucosidase, and the xylanase.

In some embodiments, the present teachings provide a method of making lactic acid comprising; fermenting a feedstock with Lactobacillus rhamnosus in a fermentation broth to make lactic acid, wherein the fermenting comprises a TrTG, wherein the lactic acid has a higher yield compared to a control fermentation lacking the TrTG, and wherein the fermentation broth has reduced viscosity compared to a control fermentation lacking the TrTG. In some embodiments, the feedstock comprises pure glucose or sugar syrup.

In some embodiments, the present teachings provide a method of making lactic acid comprising; fermenting a feedstock with Lactobacillus rhamnosus in a fermentation broth to make lactic acid, wherein the fermenting comprises a betaglucanase, xylanase and cellulase enzyme complex, wherein the lactic acid has a higher yield compared to a control fermentation lacking the betaglucanase, xylanase and cellulase enzyme complex, and wherein the fermentation broth has reduced viscosity compared to a control fermentation lacking the betaglucanase, xylanase and cellulase enzyme complex.

More generally, the present invention involves adding additional enzymes during fine-chemicals fermentation. Increasing product yield, decreasing antifoam consumption, improving downstream efficiency. These objectives can be largely achieved by adding additional enzymes, especially a transglycosidase activity containing enzyme preparation. Without being bound by theory, the present inventors have found out that during bio-refinery fermentation, with the help of additional enzymes, beneficial effects may be found, including but not limited to, strain stimulation, accelerated fermentation rate, increased fermentation product yield, reduction in final residual sugars, decreased consumption of antifoam, decreased fermentation broth viscosity, and/or increased precipitation/settling speed.

### BRIEF DESCRIPTION OF THE FIGURES

FIG. 1 depicts the effect of Transglucosidase on the settling of insolubles of fermentation broth using glucose syrup as substrate.

FIG. 2 depicts the effect of TrTGL2000 on settling of insolubles of fermentation broth using pure glucose as substrate.

FIG. 3 depicts the effect of TrGA on settling of insolubles of fermentation broth using glucose syrup as substrate.

FIGS. 4A-B depict the effect of TrTG L2000 on settling of insolubles of fermentation broth using pure glucose as substrate, strain Corynebacterium glutamicum 10178.

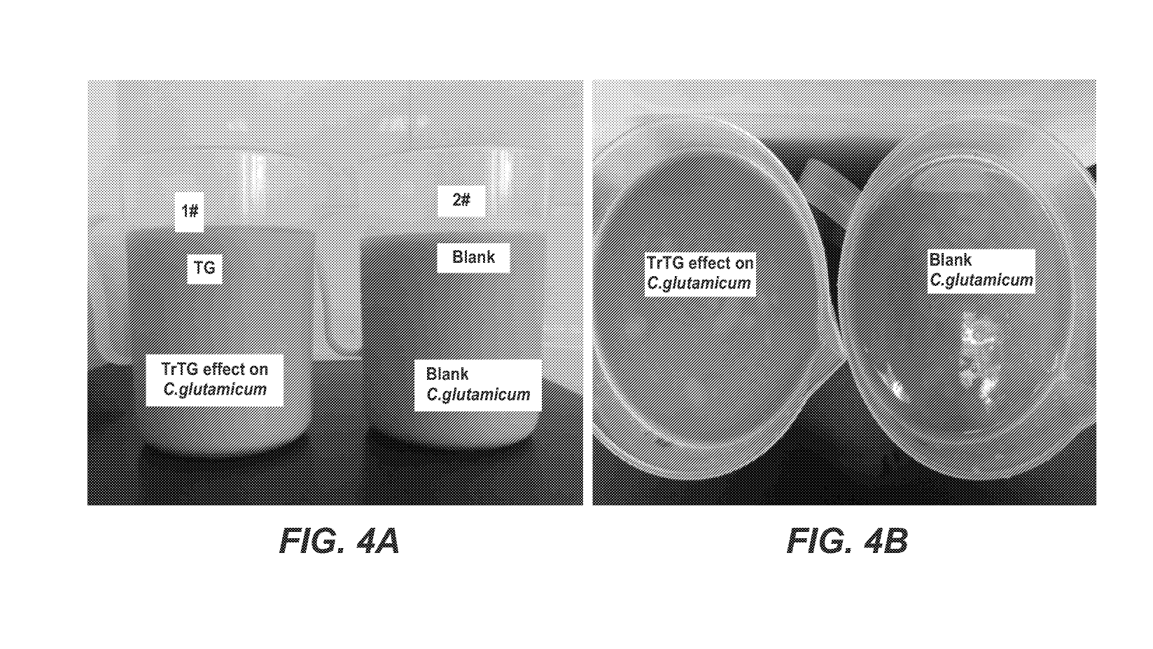

FIGS. 5A-C depict the effect of TrTG L2000 on foaming broth using pure glucose as substrate, strain Corynebacterium glutamicum SP 10238.

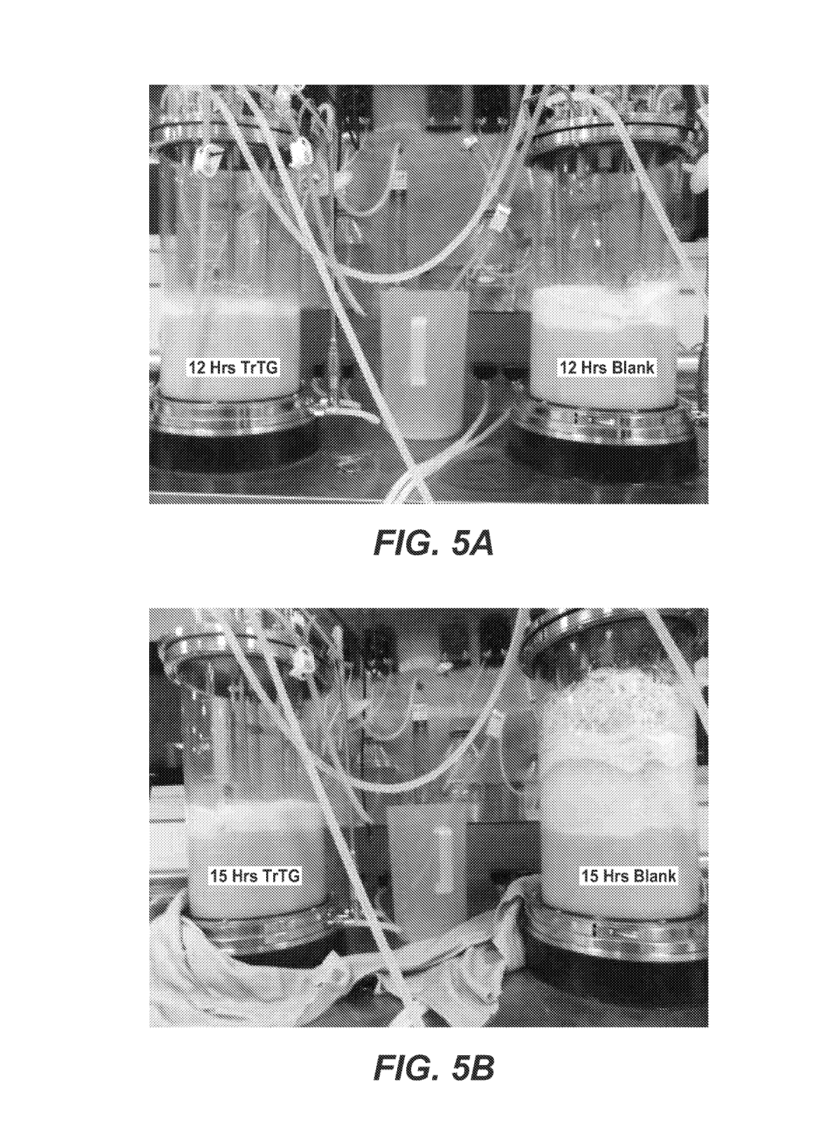

FIG. 6 depicts the effect of ACCELLERASE DUET on foaming broth using pure glucose as substrate, strain Corynebacterium glutamicum SP 10238.

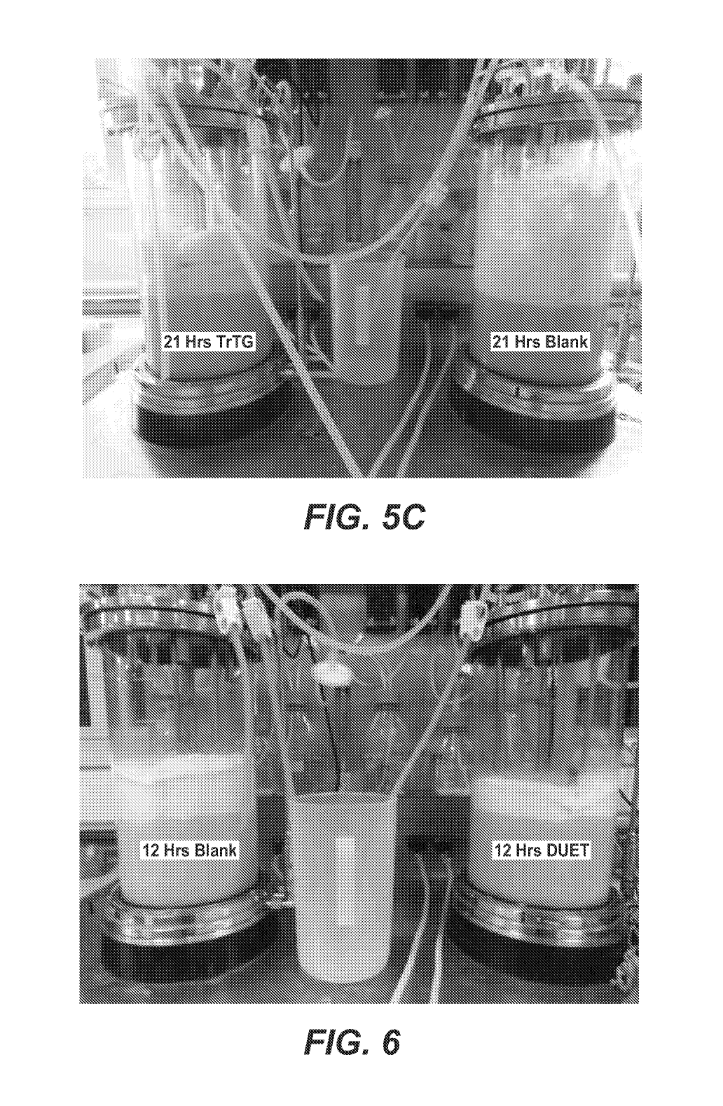

### DEFINITIONS

As used herein, the term “transglycosidase” refers to any enzyme or enzyme-containing preparation capable of transferring a monosaccharide moiety, as defined below, from one molecule to another. The term ‘transglucosidase’ is used when the monosaccharide moiety is a glucose moiety. In one embodiment, the transglycosidase enzyme is a transglucosidase enzyme. In one embodiment, the transglycosidase is classified in enzyme classification (E.C.) 3.2.1.24. In one embodiment, the transglycosidase enzyme is classified in Glycoside Hydrolase Family 31 of the Carbohydrate-Active Enzymes (CAZy) database. This database is described at http://www.cazy.org/ and in Coutinho, P. M. & Henrissat, B. (1999). This classification system is based on structural and sequence features rather than substrate specificity: as there is a direct relationship between sequence and folding similarities; such a classification: (i) reflects the structural features of these enzymes better than their sole substrate specificity, (ii) helps to reveal the evolutionary relationships between these enzymes, and (iii) provides a convenient tool to derive mechanistic information. In this classification, glycoside hydrolases (EC 3.2.1.-) are a widespread group of enzymes which hydrolyse the glycosidic bond between two or more carbohydrates or between a carbohydrate and a non-carbohydrate moiety. Further description of the classification of glycoside hydrolases (glycosidases and transglydosidases can be found in Henrissat B (1991), Henrissat B, Bairoch A (1993), Henrissat B, Bairoch A (1996), Davies G, Henrissat B (1995) and Henrissat B, Davies G J (1997)). In one embodiment, the transglycosidase enzyme is obtainable or is obtained from a living organism. Suitable transglycosidase enzymes are of bacterial or fungal origin. Preferred are transglycosidase enzymes of fungal origin. In one embodiment, the transglycosidase enzyme is of fungal origin or has at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity with the mature transglucosidase enzyme of fungal origin that is SEQ ID NO: 1 (herein referred to as a “TrTG”, since the transglucosidase was made by expression in Trichoderma, as described generally in WO 09/114380).

SEQ ID NO: 1
Asqsllsttapsqpqftipasadvgaglianiddpqaadaqsvcpgykas
kvqhnsrgftaslqlagrpcnvygtdvesltlsveyqdsdriniqilpth
vdstnaswyflsenlvprpkaslnasysqsdlfvswsnepsfnfkvirka
tgdalfstegtvlvyenqfiefvtalpeeynlyglgehitqfrlqrnani
tiypsddgtpidqnlygqhpfyldtryykgdrqngsyipvksseadasqd
yislshgvflmshgleillrsqkliwrtlgggidltfysgpapadvtrqy
ltstvglpamqqyntlgfhqcrwgynnwsdladvvanfekfeipleyiwt
didymhgyrnfdndqhrfsysegdeflsklhesgryyvpivdaalyipnp
enasdayatydrgaaddvflknpdgslyigavwpgytvfpdwhhpkavdf
wanelviwskkvafdgvwydmsevssfcvgscgtgnitlnpahpsfllpg
epgdiiydypeafnitnateaasasagassqaaatatttstsysylrttp
tpgvrnvehppyvinhdqeghdlsvhayspnathydgveeydvhglyghq
glnatyqgllevwshkrrpfiigrstfagsgkwaghwggdnyskwwsmyy
sisqalsfslfdipmfgadtcgfngnsdeelcnrwmqlsaffpfyrnhne
lstipqepyrwasvieatksamriryailpyfytlfdlahttgstvmral
swefpndptlaavetqfmvgpaimvvpvleplvntvkgvfpgvghgevwy
dwytqaavdakpgvnttisaplghipvyvrggnilpmqepalttrearqt
pwallaalgsngtasgqlylddgesiypnatlhvdftasrssirssaqgr
wkemplanvtvlgvnkepsavtlngqavfpgsvtynstsqvlfvgglqni
tkggawaenwvlew

A nucleic acid sequence encoding this protein is found in SEQ ID NO: 2.

SEQ ID NO: 2
tccaccactgccccttcgcagccgcagtttaccattcctgcttccgcaga
tgtcggtgcgcagctgattgccaacatcgatgatcctcaggctgccgacg
cgcagtcggtttgtccgggctacaaggcttcaaaagtgcagcacaattca
cgtggattcactgccagtcttcagctcgcgggcaggccatgtaacgtata
cggcacagatgttgagtccttgacactgtctgtggagtaccaggattcgg
atcgactgaatattcagattctccccactcatgttgactccacaaacgct
tcttggtactttctttcggaaaacctggtccccagacccaaggcttccct
caatgcatctgtatcccagagcgacctttttgtgtcatggtcaaatgagc
cgtcgttcaatttcaaggtgatccgaaaggctacaggcgacgcgcttttc
agtacagaaggcactgtgctcgtatatgagaatcagttcatcgaatttgt
gaccgcgctccctgaagaatataacttgtatggccttggggagcatatca
cgcaattccgcctccagagaaatgctaatctgaccatatatccttcggat
gatggaacacctattgaccagtgagtactgatatcccgcccgtatcttct
ggttctactcttgaaacttactcgtcctagaaacctctacggccaacatc
ccttctatctggatacaagatattacaaaggagataggcagaatgggtct
tatattcccgtcaaaagcagcgaggctgatgcctcgcaagattatatctc
cctctctcatggcgtgtttctgaggaactctcatggacttgagatactcc
tccggtctcaaaaattgatctggcggaccctaggtggaggaatcgatctc
accttctactcaggccccgccccggccgatgttaccaggcaatatcttac
cagcactgtgggattaccggccatgcagcaatacaacactcttggattcc
accaatgtcgttggggctacaacaactggtcggatctggcggacgttgtt
gcgaactttgagaagtttgagatcccgttggaatatatctggtgcgtatt
gtactggtttatggtatctcaaaacagtctaacaggcacttaggaccgat
attgactacatgcacggatatcgcaactttgacaacgatcaacatcgctt
ttcctacagtgagggcgatgaatttctcagcaagctacatgagagtggac
gctactatgtacccattgttgatgcggcgctctacattcctaatcccgaa
aatgcctctgatgcgtaagtgtctagtgacaaattatattactgcctgta
tgctaattagcgatacagatacgctacgtatgacagaggagctgcggacg
acgtcttcctcaagaatcccgatggtagcctctatattggagccgtttgg
ccaggatatacagtcttccccgattggcatcatcccaaggcagttgactt
ctgggctaacgagcttgttatctggtcgaagaaagtggcgttcgatggtg
tgtggtacgacatgtctgaagtttcatccttctgtgtcgggagctgtggc
acaggtaacctgactctgaacccggcacacccatcgtttcttctccccgg
tgagcctggtgatatcatatatgattacccagaggctttcaatatcacca
acgctacagaggcggcgtcagcttcggcgggagcttccagtcaggctgca
gcaaccgcgaccaccacgtcgacttcggtatcatatctgcggacaacgcc
cacgcctggtgtccgcaatgttgagcacccaccctatgtgatcaaccatg
accaagaaggccatgatctcagtgtccatgcggtgtcgccgaatgcaacg
catgttgatggtgttgaggagtatgatgtgcacggtctctacggacatca
aggattgaacgctacctaccaaggtctgcttgaggtctggtctcataagc
ggcggccatttattattggccgctcaaccttcgctggctctggcaaatgg
gcaggccactggggcggcgacaactattccaaatggtggtccatgtacta
ctccatctcgcaagccctctccttctcacttttcggcattccgatgtttg
gtgcggacacctgtgggtttaacggaaactccgatgaggagctctgcaac
cgatggatgcaactgtccgcattcttcccattctaccgaaaccacaatga
gctctccacaatcccacaggagccttatcggtgggcttctgttattgaag
caaccaagtccgccatgagaattcggtacgccatcctaccttacttttat
acgttgtttgacctggcccacaccacgggctccactgtaatgcgcgcact
ttcctgggaattccctaatgacccaacattggctgcggttgagactcaat
tcatggttgggccggccatcatggtggtcccggtattggagcctctggtc
aatacggtcaagggcgtattcccaggagttggacatggcgaagtgtggta
cgattggtacacccaggctgcagttgatgcgaagcccggggtcaacacga
ccatttcggcaccattgggccacatcccagtttatgtacgaggtggaaac
atcttgccgatgcaagagccggcattgaccactcgtgaagcccggcaaac
cccgtgggctttgctagctgcactaggaagcaatggaaccgcgtcggggc
agctctatctcgatgatggagagagcatctaccccaatgccaccctccat
gtggacttcacggcatcgcggtcaagcctgcgctcgtcggctcaaggaag
atggaaagagaggaacccgcttgctaatgtgacggtgctcggagtgaaca
aggagccctctgcggtgaccctgaatggacaggccgtatttcccgggtct
gtcacgtacaattctacgtcccaggttctctttgttggggggctgcaaaa
cttgacgaagggcggcgcatgggcggaaaactgggtattggaatgg

In some embodiments, the transglycosidase enzyme originates from an Aspergillus species, especially an Aspergillus species selected from the group consisting of Aspergillus niger (herein referred to as an “A. niger TG”), Aspergillus awamori, Aspergillus terreus, Aspergillus oryzae, Aspergillus nidulans, Aspergullus fumigatus and Aspergillus clavatus or has at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity with a transglucosidase enzyme originating from an Aspergillus species. In one embodiment, the transglycosidase enzyme is Aspergillus niger a sequence having at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, sequence identity therewith. The transglycosidase enzyme may be post-translationally modified, for example by cleavage of a signal sequence or by glycosylation.

As used herein the term “foam reduction” refers to the reduction of at least 1 centimeter of foam after 12 hours relative to a blank foam height condition as assessed according to the general conditions described in Example 6. In some embodiments, the foam reduction comprises at least 1 cm, 2 cm, 3 cm, 4 cm, or 5 cm relative to a control fermentation lacking the transglycosidase.

As used herein the term “viscosity reduction” refers to a decrease in viscosity (mpas) as measured after 12 hours relative to a control group as assessed according to the general conditions described in Example 8. In some embodiments, the viscosity reduction comprises at least 1%, 5%, 10%, 20%, 25%, 30%, 40%, 50%, or 60% reduction relative to a control fermentation lacking the transglycosidase.

As used herein, the term “pure glucose” refers to at least 95% glucose.

As used herein, the term “pure sucrose” refers to at least 95% sucrose.

As used herein, the term “feedstock” refers to any carbon source that can be utilized by a fermenting microorganism to make a fermentation product. Illustrative feedstocks include pure glucose, pure sucrose, granular starch, liquefied starch, and any of a variety of cellulosic materials.

As used herein, “fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

As used herein, “fermentation product” refers to any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, tryptophan, and threonine); a gas (e.g., methane, hydrogen (H2), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-Dgluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); 1-3 propane diol, and polyketide. The fermentation product can also be protein as a high value product.

In addition to those explicitly exemplified herein, one of skill in the art will appreciate the following organisms can be employed to make the corresponding fermentation product. For example, lysine can be made with a bacterium selected from the group coryneform bacteria, Entobacteriaceae and Bacillus, as well as Corynebacterium glutamicum, Corynebacterium efficiens, Corynebacterium callunae, Corynebacterium thermoaminogenes, Corynebacterium ammoniagenes, and E. Coli. MSG can be made with Corynebacterium Glutamicum or E. Coli. Lactic acid can be made with a Bacteria based process, including Bacillus Smithii, Bacillus Coagulans, Bacillus Thermoamylovorans, Geobacillus stearothemophilis, as well as a Yeast based process, including Candida, Saccharomyces, Shizosaccharomyes, Kluyveromyces, Pichia, Issachenkia or Hansenula.

### DETAILED DESCRIPTION OF THE INVENTION

### Materials

Microorganism for L-Lysine Fermentation

Examples of fermenting organisms include Brevibacterium flavum 20879 and Corynebacterium glutamicum 10178, purchased from CICC (China Center of Industrial Culture Collection).

Microorganism for L-Glutamic Acid Fermentation

Example of fermenting organisms include Corynebacterium glutamicum sp 10238, purchased from CICC (China Center of Industrial Culture Collection).

Microorganism for L-Lactic Acid Fermentation

Example of fermenting organisms include Lactobacillus rhamnosus, purchased from CGMCC (China General Microbiological Culture Collection Center).

Microorganism Culture Condition

Agar Seed Preparing

Lysine fermentation microorganism agar plate medium for use in this invention includes: glucose 5 g/L, NaCl: 5 g/L, beef extract 10 g/L, peptone 10 g/L, agar 20 g/L, pH 7.0;

Glutamic acid microorganism agar plate medium for use in this invention includes: glucose 5 g/L, NaCl: 5 g/L, beef extract 10 g/L, peptone 10 g/L, agar 20 g/L, pH 7.0;

Lactic acid microorganism agar plate medium for use in this invention includes: Casein 10.0 g, Beef extract 10.0 g, Yeast extract 5.0 g, Glucose 5.0 g, Sodium acetate 5.0 g, Diammonium citrate 2.0 g, Tween 80 1.0 g, K₂HPO₄ 2.0 g, MgSO₄.7H₂O 0.2 g, MnSO₄.H₂O 0.05 g, agar 20 g, Distilled water 1.0 L, pH6.8.

The agar plate medium is sterilized at 121° C. holding for 20 min(vertical heating pressure steam sterilizer, LDZM-80KCS, Shanghai Shenan medical instrument factory) then dispense to plate, after solidification, cultivate in room temperature for 24 hrs for contamination test. The bacterial is transferred into agar plate at clean bench (OptiMair®, Ecso Laminar Flow cabinet) and cultivate in BINDER incubator 24 hrs.

### Fermentation Seed Preparing

Lysine fermentation microorganism seed medium for use in this invention includes: Glucose 25 g/L, CSLP (Corn Steep Liquor Powder) 24 g/L, (NH₄)₂SO₄ 5 g/L, KH₂PO₄ 1 g/L, MgSO₄.7H₂O 0.5 g/L, CaCO₃ 15 g/L, pH 7.0. Prepare 1 L seed medium, adjust pH according to the Prescription above, then dispense 60 mL to every 500 mL baffled flask for better oxygen dissolve, then sterilized at 121° C. holding for 20 min, after cool down to room temperature, transfer the biomass from agar plate to flasks, then cultivate on a NBS rotary shaker at 130 rpm, 30° C., 8˜10 hrs, maintain the OD above 0.8, check the strain through microscope to make sure the bacterial shape was “v-shape” and without contamination.

Glutamic acid fermentation microorganism seed medium for use in this invention includes: Glucose 25 g/L, CSLP 24 g/L, K₂HPO₄ 1.5 g/L, MgSO₄.7H₂O 0.4 g/L, urea 5 g/L, pH 7.3. Prepare 1 L seed medium, adjust pH according to the Prescription above, then dispense 60 mL to every 500 mL baffled flask for better oxygen dissolve, then sterilized at 121° C. holding for 20 min, after cool down to room temperature, transfer the biomass from agar plate to flasks, then cultivate on a NBS rotary shaker at 130 rpm, 32° C., 5˜6 hrs, maintain the OD above 0.9, check the strain through microscope to make sure the bacterial shape was “v-shape” and without contamination.

Lactic acid fermentation microorganism seed medium for use in this invention includes: Casein 10.0 g, Beef extract 10.0 g, Yeast extract 5.0 g, Glucose 5.0 g, Sodium acetate 5.0 g, Diammonium citrate 2.0 g, Tween 80 1.0 g, K₂HPO₄ 2.0 g, MgSO₄.7H₂O 0.2 g, MnSO₄.H₂O 0.05 g, pH6.8. Prepare 1 L seed medium, adjust pH according to the Prescription above, then dispense 100 mL to every 300 mL flask, then sterilized at 121° C. holding for 20 min, after cool down to room temperature, transfer the biomass from agar plate to flasks, then cultivate on a NBS rotary shaker at 150 rpm, 37° C., 14˜18 hrs, maintain the OD above 0.5, check the strain through microscope to make sure no contamination.

### Fermentation Medium Preparing

Lysine fermentation microorganism fermentation nutrient medium in 7 L jar fermentor includes: Initial glucose 80 g/L, CSLP 15 g/L, (NH₄)₂SO₄ 15 g/L, KH₂PO₄ 1 g/L, MgSO₄.7H₂O 0.5 g/L, FeSO₄.7H₂O 9.9 mg/L, MnSO₄.H₂O 6.15 mg/L, Vitamin B1 (VB1) 100 μg/L, Biotin (VH) 100 μg/L, Antifoam THIX-298 0.05 g/L. For lysine fermentation the initial working volume is 3.5 L (nutrition 2.75 L, initial glucose 0.4 L, seed broth 0.35 L), nutrition prepare: weight powder CSL (Corn Steep Liquor) 52.5 g, (NH4)₂SO₄ 52.5 g, KH₂PO₄ 3.5 g, MgSO₄.7H₂O 1.75 g, FeSO₄.7H₂O 7 mg, MnSO₄.H2O 7 mg, VB₁ 350 μg, VH 350 μg, antifoam 0.175 g, dissolved in 2.75 L water added to fermentor. Initial glucose prepare: weight 240 g glucose dissolved in water and make the total volume at 400 mL and add in one flask. High concentration glucose prepare (for feeding in the fermentation process): weight 400 g glucose dissolved in water and make the total volume at 500 mL and add in one flask using a pipe to gear into the fermentor. Antifoam prepare: weight 20 g antifoam dissolved in 80 g water and then add in one flask using a pipe to gear into the fermentor. Prepare an empty flask (used for NH₃.H₂O in the fermentation process to adjust pH) and using a pipe to gear into the fermentor, Calibrate pH electrode (METTLER TOLEDO, InPro®3030) and polarize dissolved oxygen electrode (METTLER TOLEDO) up 8 hrs, Fix the fermentor, make sure all the flasks stay along with the fermentor, and all the medium should be sterilized at 121° C. holding for 20 min, Repeat all the things above for another 7 L fermentor fermentation medium prepare. After sterilization, the fermentor should be pump into sterile air to avoid contamination; Pump the initial glucose to fermentor, and add NH₃.H₂O to the empty flask quickly, then adjust the pH to 6.5; Open the water tap, and control the temperature at 30 C by auto. Set up the stir at 200 rpm, waiting for about half an hour for all the parameters moderation, set the DO electrode at 100%; 350 mL seed broth added to the fermentor, one fermentor with enzyme and one fermentor without enzyme, begin fermentation, after seed add to fermentor the DO will decrease sharply, the agitation speed and aeration should be increased to maintain the DO at 15%˜30%; In the fermentation process, samples were withdrawn every three or four hours to measure glucose and lysine, if the glucose concentration below 30 g/L, then begin feed high concentration of glucose to maintain the glucose concentration around 30 g/L˜40 g/L.

Glutamic acid fermentation microorganism fermentation nutrient medium in 7 L jar fermentor includes: Initial glucose 90˜100 g/L, CSLP 4 g/L, MgSO₄.7H₂O 0.8 g/L·K₂HPO₄ 2 g/L, FeSO₄.7H₂O 22 mg/L, MnSO₄.H₂O 22 mg/L, Vitamin B₁ (VB₁) 0.2 mg/L, antifoam THIX-298 0.1 g/L. For glutamic acid fermentation the initial working volume can be 2.5˜2.7 L, feeding glucose can be 200 ml˜500 ml. For example if glutamic acid fermentation the initial working volume is 2.5 L (nutrition 1.8 L, initial glucose 0.4 L, seed broth 0.30 L), nutrition prepare: weight powder CSL 10.0 g, MgSO₄.7H₂O 2.0 g, KH₂PO₄ 5 g, FeSO₄.7H₂O 55 mg, MnSO₄.H₂O 55 mg, VB₁ 500 μg, antifoam 0.25 g, dissolved in 1.8 L water added to fermentor. Initial glucose prepare: weight 250 g glucose dissolved in water and make the total volume at 400 mL and add in one flask. High concentration glucose prepare 770 g/L (for feeding in the fermentation process): weight 385 g glucose dissolved in water and make the total volume at 500 mL and add in one flask using a pipe to gear into the fermentor. Antifoam prepare: weight 40 g antifoam dissolved in 160 g water and then add in one flask using a pipe to gear into the fermentor. Prepare an empty flask (used for NH₃.H₂O in the fermentation process to adjust pH) and using a pipe to gear into the fermentor, Calibrate pH electrode (METTLER TOLEDO, InPro®3030) and polarize dissolved oxygen electrode (METTLER TOLEDO) up 8 hrs, Fix the fermentor, make sure all the flasks stay along with the fermentor, and all the medium should be sterilized at 121° C. holding for 20 min, Repeat all the things above for another 7 L fermentor fermentation medium prepare. After sterilization, the fermentor should be pumped into sterile air to avoid contamination; Pump the initial glucose to fermentor, and add NH₃.H₂O to the empty flask quickly, then adjust the pH to 7.0-7.1; Open the water tap, and control the temperature at 30° C. by auto. Set up the stir at 200 rpm, waiting for about half an hour for all the parameters moderation, set the DO electrode at 100%; 300 mL seed broth added to the fermentor, one fermentor with enzyme and one fermentor without enzyme, begin fermentation, after seed add to fermentor the DO will decrease sharply, the agitation speed and aeration should be increased to maintain the DO at 30%˜50%; In the fermentation process, after 4˜6 hrs increase fermentation temperature 1° C. every three or four hours till 38° C., samples were withdrawn every three or four hours to measure glucose and glutamic acid, if the glucose concentration below 20 g/L, then begin feed high concentration of glucose to maintain the glucose concentration around 20 g/L˜30 g/L.

Lactic acid fermentation microorganism fermentation nutrient medium in 1 L jar fermentor includes: Initial sugar 80 g/L, CSLP 40 g/L, casein 10 g/L, beef extract 10 g/L, yeast extract 10 g/L, Tween 80 1.5 g/L, MnSO₄.H₂O 0.3 g/L, calcium carbonate 20 g/, pH 6.5. For Lactic acid fermentation the initial working volume is 0.6 L (nutrition 0.5 L, initial glucose 0.05 L, seed broth 0.05˜0.10 L), nutrition prepare: weight powder corn steep liquor 20 g, casein 5.0 g, Beef extract 5.0 g, Yeast extract 5.0 g, Tween 80 0.75 g, MnSO4.H2O 0.15 g, Calcium carbonate 10 g, pH 6.5, Initial glucose prepare: weight 40 g glucose dissolved in water and make the total volume at 50 mL and add in one flask. High concentration glucose prepare (for feeding in the fermentation process): weight 100 g glucose dissolved in water and make the total volume at 150 mL and add in one flask using a pipe to gear into the fermentor. Prepare an empty or contained 50 ml distilled water flask (used for NH₃.H₂O in the fermentation process to adjust pH,) and using a pipe to gear into the fermentor, Calibrate pH electrode (METTLER TOLEDO, InPro®3030) and polarize dissolved oxygen electrode (METTLER TOLEDO) up 8 hrs, Fix the fermentor, make sure all the flasks stay along with the fermentor, and all the medium should be sterilized at 121° C. holding for 20 min, Repeat all the things above for another 1 L fermentor fermentation medium prepare. After sterilization, the fermentor should be pump into sterile air to avoid contamination; Pump the initial glucose to fermentor, and add NH₃.H₂O to the empty flask quickly, then adjust the pH to 6.5; Open the water tap, and control the temperature at 40˜45° C. by auto. Set up the stir at 200 rpm, waiting for about half an hour for all the parameters moderation, then added 50 mL seed broth to the fermentor, one fermentor with enzyme and one fermentor without enzyme, begin fermentation, In the fermentation process, pH was adjusted automatically by adding additional NH₃.H₂O or diluted NH₃.H₂O. Samples were withdrawn every three or four hours to measure sugar and lactic acid level analyzed by HPLC.

As used herein, CSLP refers to the spray-dried Corn Steep Liquor Powder, which is widely used in Microbial Fermentation, purchased from Roquette Co., Ltd. The glucose syrup is Liquid Glucose purchased from Xiwang Group Co., Ltd. HPAED analysis results indicated greater than 85% glucose expressed as mg/L. AntifoamTHIX-298 was bought from YanTai Thinking Finechem Technology Co. Ltd in Shandong province. The 7 L jar fermentor was purchased from KOBIOTECH CO., LTD. The 1 L jar fermentor was purchased from APPLITECH CO., LTD. Unless otherwise specified, other chemicals are purchased from Sino-pharm Co., Ltd; beef extract, peptone, casein, agar are BR grade, others are AR grade.

### Enzymes

Transglucosidase L-2000 (product of DuPont Industrial Biosciences, 2013): purified D-glucosyltransferase (transglucosidase, EC 2.4.1.24) free from glucoamylase activity. Transglucosidase L-2000 is produced through controlled fermentation, using a genetically modified strain of Trichoderma reesei.

Transglucosidase L “Amano” (manufactured by Amano Pharmaceutical Co., Ltd.),): the Aspergillus niger glucosyltransferase. This enzyme is an α-glucosidase (EC 3.2.1.20), and is capable of hydrolyzing its substrate from the non-reducing end of the molecule to release glucose molecules. Also, under high substrate concentration, this enzyme is capable of performing a glycosyl transfer reaction rather than the hydrolysis of the substrate. Because of this glycosyl transfer capability, this enzyme is also referred as transglucosidase.

Optimash® BG (Genencor International, Inc): Viscosity Reducing Enzyme, High activity betaglucanase, xylanase and cellulase enzyme complex for rapid viscosity reduction, made in a modified Trichoderma host in which CBH1 and CBH2 cellobiohydrolases I and II) are deleted, and EGII (endoglucanase, a beta glucanase) is over-expressed by at least 50% relative to wild type, as taught for example in WO2A118257.

TrGA (Genencor International, Inc): Glucoamylase enzyme produced by a non-pathogenic, non-toxigenic strain of Trichoderma reesei, which is genetically modified to over express a native T. reesei glucoamylase enzyme (see for example U.S. Pat. No. 7,413,887)

ACCELLERASE® DUET (Genencor International, Inc): produced with a genetically modified strain of Trichoderma reesei, that contains not only exoglucanase, endoglucanase, β-glucosidase, but also includes xylanases and other hemicellulases, as commercially available from Danisco US Inc, DuPont Industrial Biosciences, in 2013.

### Methods

### Sugar Content Determination by HPAED

Sugar analysis was performed on a Dionex ICS-5000 ion chromatography (Sunnyvale, Calif., USA) composed of a GP50 gradient pump, an ED 40 electrochemical detector including a pulsed amperometric detection cell made of a gold electrode and a pH-Ag/AgCl reference electrode. Separation was performed on a CarboPak™ PA200 column (3*250 mm) with a guard column (3*50 mm). The flow rate was 0.5 ml/min on CarboPak™ PA200 column, and column temperature was 30° C. Sample injection volume was 25 μl. The gradient for CarboPak™ PA200 column used was: 0-5 min, 100 mM NaOH, 0-40 mM NaAc; 5-60 min, 100 mM NaOH, 40-500 mM NaAc; 60-70 min, 100 mM NaOH. Pulsed amperometric detection was used as detector, and following pulse potentials and durations were used: E1=0.1V, t1=400 ms; E2=−2V, t2=20 ms; E3=0.6V, t3=10 ms; E4=−0.1V, t4=70 ms. Data acquisition and integration were performed using Chromeleon 6.8 workstation.

### Fermentation Biomass Determination by Spectrophotometer

Bio-mass analysis was performed on SHIMADZU UV-1700 Spectrophotometer. Fermentation broth was diluted 25 times with 1 mol/L Hydrochloric acid, and then checked the OD (Optical Density) value at 562 nm for lysine or 620 nm for glutamic acid.

### Residual Glucose and Lysine/Glutamic Acid Content Determination by SBA40C Bio-Sensor

Both residual glucose and lysine yield were performed on SBA-40C bio-sensor (Shandong Academy of Sciences Institute of Biology). The fermentation broth was diluted 100 times with purified water, before analyzing sample calibration was required. Injected 25 μl standard sample (100 mg/dL glucose and 100 mg/dl lysine stock solution) several times to complete the calibration. Injected 25 μl sample and record the data showed on the screen. Each time of calibration 10 samples can be analyzed. If the sample number was more than 10, re-calibration step was performed.

### Lactic Acid Content Determination by HPLC(High Pressure Liquid Chromatographic):

HPLC method using an Agilent 1100, Column specification: BIO-RAD Aminex HPX-87H or Rezex ROA-organic acid. Method of analysis: ESTD. Details of the analysis: Mobile phase: 0.005 mol/L H₂SO₄. Sample was withdrawn and diluted 10 times, and filtered using 0.45 μm filter membrane. Other HPLC parameters: Injection volume: 20 μl; Pump flow: 0.6 ml/min; Column thermostat temperature: 60° C.; RID, optical unit temperature: 35° C.

### Fermentation Broth Viscosity Determination by HAAKE VT550

Viscosity analysis was performed on HAAKE VT550 viscometer with FL-10 spindle in DC30-K10 Refrigerated Circulator water bath (all from Thermo Scientific). Weighed out 100 grams fermentation broth into HAAKE tube, using FL-10 spindle and run fixed share rate A 10 [1/s] fixed temperature 30° C. process 10 min. When the process was terminated, poured the broth and cleaned the tube to run another batch. Viscosity data were recorded by HAAKE workstation.

### Natural Insoluble Solids Settling Observation

Poured the fermentation broth into a beaker and covered it with wrap, hold it in 4-8° C. refrigerator 18 hrs, and then took a picture.

### Example 1

### Comparison of Commercial Transglucosidase Activity Containing Products on Lysine Fermentation Using Sugar Syrup as Substrate: Brevibacterium flavum

Bacterial biomass collected from an agar plate transferred into seed culture medium in 500 mL flasks. Cultivate on a NBS rotary shaker at 130 rpm and 30° C., and the working volume 60 mL/500 mL flask, using baffled flask, cultivate about 10 hrs and maintain the OD above 0.8. The fermentation medium was sterilized at 121° C. for 12 min, cooled down the temperature to 30° C., add 80 g/L initial glucose to the fermentor, adjust the pH of the medium to 6.5 by ammonium hydroxide. 0.75 g TrTG L-2000 (product of Genencor International, Inc.) and Transglucosidase™ L “Amano” (manufactured by Amano Pharmaceutical Co., Ltd., Japan) with 300 mL seed broth (10% v/v) was added to fermentation medium. Temperature was maintained at 30° C., and pH was controlled at 6.8˜7.0 by addition of ammonium hydroxide, DO was maintained at 10%˜25% by adjusting aeration and agitation speed. In the fermentation process, samples were withdrawn every three or four hours to measure glucose and maintain at suitable concentration (30 g/L˜40 g/L) by feeding high concentration of glucose, at 8 hrs and 32 hrs added 0.75 g TrTG and AMANO TG respectively.

TABLE 1
Effect of Transglucosidase on lysine fermentation yield, residual
glucose at the end of fermentation and viscosity of fermentation
broth using glucose syrup as substrate

| | Viscosity | | Residual | | Lysine•HCl | |
|---|---|---|---|---|---|---|
| | (mpas) | | glucose (g/L) | | (g/L) | |
| | | With | | With | | With |
| | BK | Enzyme | BK | Enzyme | BK | Enzyme |
| TrTG @ 3.8 Kg/MT | 70.26 | 21.62 | 11 | 2 | 51.3 | 68.8 |
| AMANO TG | 54.04 | 81.07 | 16 | 2 | 37.5 | 52.5 |
| @3.6 Kg/MT | | | | | | |

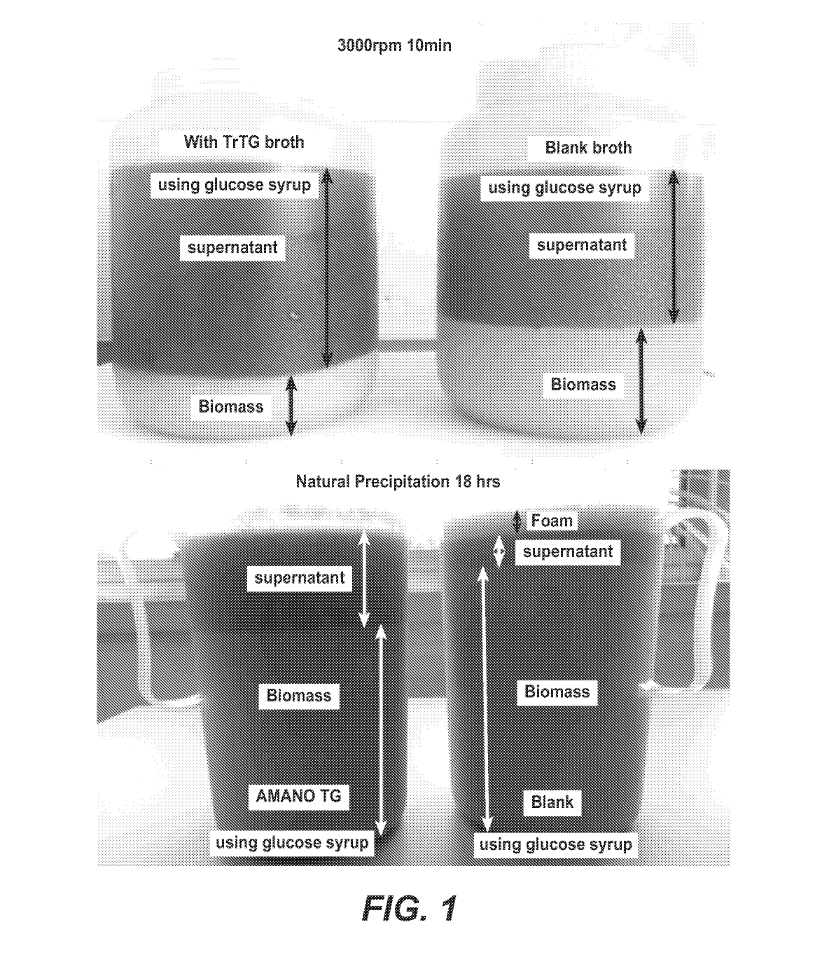

The results of Table 1 and FIG. 1 showed that additional TrTG L2000 resulted in much higher lysine. HCl yield, lower residual glucose, lower broth viscosity, and faster precipitation than blank (BK). Additionally, AMANO TG resulted in much higher lysine.HCl yield, lower residual glucose, and faster precipitation than blank as TrTG L2000 showed, but resulted in much higher broth viscosity than blank.

### Example 2

### TrTG L2000 Effect on Lysine Fermentation Using Pure Glucose as Substrate: Brevibacterium flavum

Bacterial biomass collected from an agar plate transferred into seed culture medium in 500 mL flasks. Cultivate on a NBS rotary shaker at 130 rpm and 30° C., and the working volume 60 mL/500 mL flask, using baffled flask, cultivate about 10 hrs and maintain the OD above 0.8. The fermentation medium was sterilized at 121° C. for 12 min, cooled down the temperature to 30° C., add 80 g/L initial glucose to the fermentor, adjust the pH of the medium to 6.5 by ammonium hydroxide. 0.75 g TrTG L-2000 (product of Genencor International, Inc.) and with 300 mL seed broth (10% v/v) was added to fermentation medium. Temperature was maintained at 30° C., and pH was controlled at 6.8˜7.0 by addition of ammonium hydroxide, DO was maintained at 10%˜25% by adjusting aeration and agitation speed. In the fermentation process, samples were withdrawn every three or four hours to measure glucose and maintain at suitable concentration (30 g/L˜40 g/L) by feeding high concentration of glucose, at 8 hrs and 32 hrs added 0.75 g TrTG L2000 respectively.

TABLE 2
Effect of TrTG L 2000 on lysine fermentation yield, residual
glucose at the end of fermentation and viscosity of fermentation
broth using pure glucose as substrate

| | Viscosity | | Residual | | Lysine•HCl | |
|---|---|---|---|---|---|---|
| | (mpas) | | glucose (g/L) | | (g/L) | |
| | | With | | With | | With |
| | BK | Enzyme | BK | Enzyme | BK | Enzyme |
| TrTG @3.7 Kg/MT | 54.04 | 21.62 | 1 | 1 | 37.5 | 46.3 |

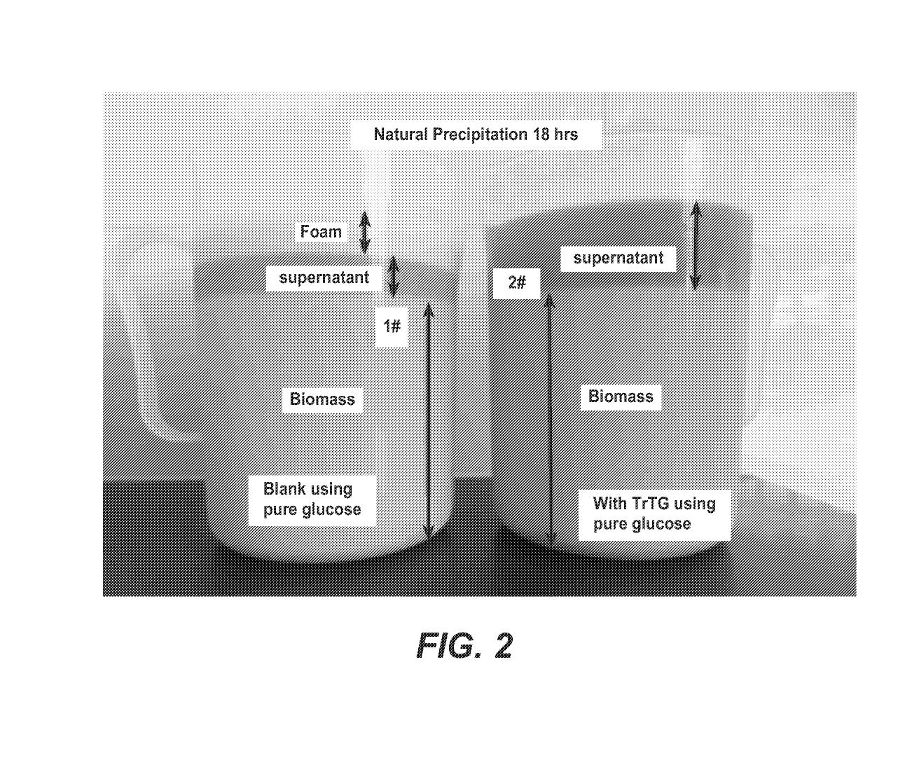

Table 2 and FIG. 2 showed that additional TrTG L2000 resulted in much higher lysine.HCl yield, lower residual glucose, lower broth viscosity, and faster precipitation than blank.

### Example 3

### Effect of TrGA on Lysine Production Using Sugar Syrup as Substrate: Brevibacterium flavum

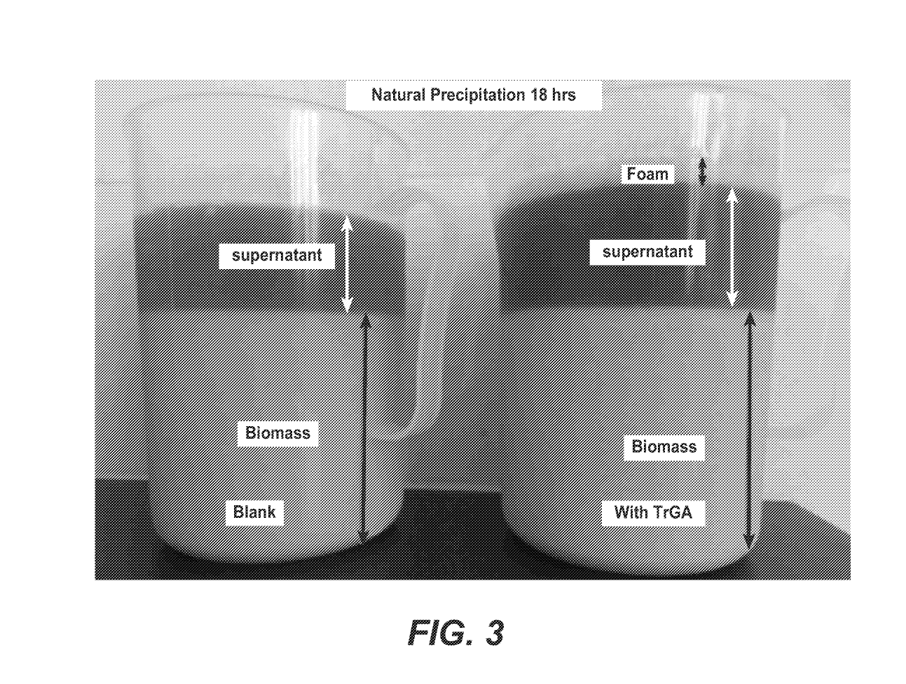

This experiment assessed whether TG was likely responsible for the improvements, or if residue from the Trichoderma production strain is likely responsible. Bacterial biomass collected from an agar plate transferred into seed culture medium in 500 mL flasks. Cultivate on a NBS rotary shaker at 130 rpm and 30° C., and the working volume 60 mL/500 mL flask, using baffled flask, cultivate about 10 hrs and maintain the OD above 0.8. The fermentation medium was sterilized at 121° C. for 12 min, cooled down the temperature to 30° C., add 80 g/L initial glucose to the fermentor, adjust the pH of the medium to 6.5 by ammonium hydroxide. Controlled fermentation pH at 6.8˜7.0 by addition of ammonium hydroxide, DO was maintained at 10%˜25% by adjusting aeration and agitation speed. In the fermentation process, samples were withdrawn every three or four hours to measure glucose and maintain at suitable concentration (30 g/L˜40 g/L) by feeding glucose syrup (500 g/400 ml). TrGA was added at the beginning of the fermentation with 0.715 g and 8 h with 0.715 g, so the total TrGA was 2.4 Kg/MT ds glucose. After fermentation, check the Lysine production, residual sugar, broth viscosity and precipitation (Table.3, FIG. 3)

TABLE 3
Effect of TrGA on lysine fermentation yield, residual glucose
at the end of fermentation and viscosity of fermentation
broth using glucose syrup as substrate

| | Viscosity | | Residual | | Lysine•HCl | |
|---|---|---|---|---|---|---|
| | (mpas) | | glucose (g/L) | | (g/L) | |
| | | With | | With | | With |
| | BK | Enzyme | BK | Enzyme | BK | Enzyme |
| TrGA @ 2.4 kg/MT | 37.83 | 54.04 | 5 | 10 | 52.5 | 46.3 |

The results showed that additional TrGA during lysine fermentation increased the broth viscosity and residual glucose but decreased Lysine.HCl production. The decrease in lysine production is indicated by comparison to the blank, which is lower. Without being bound by theory, this observation supported the hypothesis that TrTG is responsible for the improved lysine fermentation, and that residue from the Trichoderma expression host may not be responsible.

### Example 4

### Effect of OPTIMASH™ BG on Lysine Production Using Pure Glucose as Substrate: Brevibacterium flavum

Bacterial biomass collected from an agar plate transferred into seed culture medium in 500 mL flasks. Cultivate on a NBS rotary shaker at 130 rpm and 30° C., and the working volume 60 mL/500 mL flask, using baffled flask, cultivate about 10 hrs and maintain the OD above 0.8. The fermentation medium was sterilized at 121° C. for 12 min, cooled down the temperature to 30° C., the initial glucose was 80 g/L, controlled pH at 6.8˜7.0 by addition of ammonium hydroxide, DO was maintained at 10%˜25% by adjusting aeration and agitation speed. In the fermentation process, samples were withdrawn every three or four hours to measure glucose and maintain at suitable concentration (30 g/L˜40 g/L) by feeding glucose solution (500 g/400 ml). OPTIMASH BG was added at the beginning of the fermentation, 16 h, and 32 h with 1.12 g, 0.8 g, 0.8 g separately, the total dosage of OPTIMASH BG addition was 4 Kg/MT ds glucose, after fermentation, checked the Lysine production, residual sugar and broth viscosity.

TABLE 4
Effect of OPTIMASH BG on lysine fermentation yield, residual glucose
at the end of fermentation and viscosity of fermentation broth using
pure glucose as substrate

| | Viscosity | | Residual | | Lysine•HCl | |
|---|---|---|---|---|---|---|
| | (mpas) | | glucose (g/L) | | (g/L) | |
| | | With | | With | | With |
| | BK | Enzyme | BK | Enzyme | BK | Enzyme |
| OPTIMASH BG @ | 31.00 | 28.90 | 25 | 4 | 32.5 | 43.8 |
| 4.0 kg/MT | | | | | | |

The results showed that additional OPTIMASH BG during lysine fermentation potentially slightly decreased broth viscosity and residual glucose, and significantly increased Lysine.HCl production.

### Example 5

### Effect of Transglucosidase on Lysine Production Using Pure Glucose as Substrate: Corynebacterium glutamicum 10178

Bacterial biomass collected from an agar plate transferred into seed culture medium in 500 mL flasks. Cultivate on a NBS rotary shaker at 130 rpm and 30° C., and the working volume 60 mL/500 mL flask, using baffled flask, cultivate about 10 hrs and maintain the OD above 0.8. The fermentation medium was sterilized at 121° C. for 12 min, cooled down the temperature to 30° C., the initial glucose was 100 g/L, controlled pH at 6.8˜7.0 by addition of ammonium hydroxide, DO was maintained at 10%˜25% by adjusting aeration and agitation speed. In the fermentation process, samples were withdrawn every three or four hours to measure glucose and maintain at suitable concentration (30 g/L˜40 g/L) by feeding glucose solution (800 g/1000 ml). Transglucosidase L-2000 (product of Genencor International, Inc.) was added at the beginning of the fermentation, 15 h, 23 h, 32 h, and 36 h with 1.4 g, 0.8 g, 0.8 g, 0.8 g and 0.8 g separately. The total dosage of Transglucosidase L-2000 was 4 Kg/MT ds glucose, after fermentation, checked the Lysine production, residual sugar, broth viscosity and broth color.

TABLE 5
Effect of TrTG on lysine fermentation yield, residual glucose at the
end of fermentation and viscosity of fermentation broth using pure
glucose as substrate, strain Corynebacterium glutamicum 10178.

| | Viscosity | | Residual | | Lysine•HCl | |
|---|---|---|---|---|---|---|
| | (mpas) | | glucose (g/L) | | (g/L) | |
| | | With | | With | | With |
| | BK | Enzyme | BK | Enzyme | BK | Enzyme |
| TrTG @ 4.0 kg/MT | 6.50 | 7.42 | 11 | 16 | 57.5 | 55.0 |

The results showed that although the color changed with additional Transglucosidase L-2000, however, the enzyme did not affect the Lysine.HCl production.

### Example 6

### Effect of Transglucosidase on Glutamic Acid Production Using Pure Glucose as Substrate: Corynebacterium glutamicum SP 10238

Bacterial biomass collected from an agar plate transferred into seed culture medium in 500 mL flasks. Cultivate on a NBS rotary shaker at 130 rpm and 32° C., and the working volume 60 mL/500 mL flask, using baffled flask, cultivate about 5˜6 hrs and maintain the OD above 0.9. The fermentation medium was sterilized at 121° C. for 12 min, cooled down the temperature to 30° C. The initial glucose was 100 g/L, controlled pH at 7.0˜7.1 by addition of ammonium hydroxide, DO was maintained at 20%˜40% by adjusting aeration and agitation speed. In the fermentation process, after 4˜6 hrs increase fermentation temperature 1° C. every three or four hours till 38° C., samples were withdrawn every three or four hours to measure glucose and glutamic acid, if the glucose concentration below 20 g/L, then begin feed high concentration of glucose to maintain the glucose concentration around 20 g/L˜30 g/L. Transglucosidase L-2000 (product of Genencor International, Inc.) was added at the beginning of the fermentation (0 h), 8 h, 16 h, 21 h with 0.9525 g, 0.9525 g, 0.3175 g, and 0.3175 g separately. The total dosage of Transglucosidase L-2000 was 4 Kg/MT ds glucose, took picture during fermentation to record foaming level changing, record the glutamic acid production, residual sugar, antifoam consumption, broth viscosity and broth color after fermentation.

TABLE 6
Effect of TrTG on Glutamic acid fermentation yield, residual glucose
at the end of fermentation and viscosity of fermentation broth using
pure glucose as substrate, strain Corynebacterium glutamicum SP 10238.

| | Viscosity | | Residual | | Glutamic | |
|---|---|---|---|---|---|---|
| | (mpas) | | glucose (g/L) | | acid (g/L) | |
| | | With | | With | | With |
| | BK | Enzyme | BK | Enzyme | BK | Enzyme |
| TrTG@4 Kg/MT | 21.62 | 5.4 | 23 | 7 | 59 | 63 |

TABLE 7
Effect of TrTG on antifoam consumption using pure glucose as
substrate, strain Corynebacterium glutamicum SP 10238.

| | | Blank | | |
|---|---|---|---|---|
| | With TrTG | broth | | |
| | broth height | height | With TrTG Foam | Blank Foam |
| Time (hrs) | (cm) | (cm) | height (cm) | height (cm) |
| 0 | 13 | 13 | 0 | 0 |
| 4 | 13 | 13 | 0 | 0 |
| 6 | 13 | 13 | 0 | 3 |
| 12 | 13 | 13 | 2 | 4~5 |
| 13 | 13 | 13 | 2 | 8 (begin to add |
| | | | | antifoam) |
| 15 | 13 | 13 | 3 | 15 |
| 21 | 14 | 14 | 7 (begin to add | 15 |
| | | | antifoam) | |

| | | |
|---|---|---|
| 45 hrs total 20% antifoam | 40 | 100 |
| consumption (ml) | | |

These results showed that additional TrTG during glutamic acid fermentation decreased broth viscosity and residual glucose and significantly increased glutamic acid production, and at same time reduced the foaming formation and decreased antifoam consumption at least 50%.

### Example 7

### Effect of ACCELLERASE™ DUET on Glutamic Acid Production Using Pure Glucose as Substrate: Corynebacterium glutamicum SP 10238

Bacterial biomass collected from an agar plate transferred into seed culture medium in 500 mL flasks. Cultivate on a NBS rotary shaker at 130 rpm and 32° C., and the working volume 60 mL/500 mL flask, using baffled flask, cultivate about 5˜6 hrs and maintain the OD above 0.9. The fermentation medium was sterilized at 121° C. for 12 min, cooled down the temperature to 30° C., the initial glucose was 92.5 g/L, controlled pH at 7.0˜7.1 by addition of ammonium hydroxide, DO was maintained at 30%˜50% by adjusting aeration and agitation speed. In the fermentation process, after 4˜6 hrs increase fermentation temperature 1° C. every three or four hours till 38° C., samples were withdrawn every three or four hours to measure glucose and glutamic acid, if the glucose concentration below 20 g/L, then begin feed high concentration of glucose to maintain the glucose concentration around 20 g/L˜30 g/L. ACCELLERASE DUET (product of DuPont Industrial Biosciences, 2013) was added at the beginning of the fermentation (0 h), 8 h, 16 h, with 0.808 g, 0.808 g, and 0.202 g separately. The total dosage of ACCELLERASE DUET was 4.5 Kg/MT ds glucose, took picture during fermentation to record foaming level changing, record the glutamic acid production, residual sugar, antifoam consumption, broth viscosity and broth color after fermentation.

TABLE 8
Effect of ACCELLERASE ™ DUET on Glutamic acid fermentation
yield, residual glucose at the end of fermentation and viscosity of
fermentation broth using pure glucose as substrate, strain
Corynebacterium glutamicum SP 10238.

| | Viscosity | | Residual | | Glutamic | |
|---|---|---|---|---|---|---|
| | (mpas) | | glucose (g/L) | | acid (g/L) | |
| | | With | | With | | With |
| | BK | Enzyme | BK | Enzyme | BK | Enzyme |
| ACCELLERASE | 27.02 | 5.4 | 7 | 3 | 47 | 50 |
| DUET @ | | | | | | |
| 4.5 kg/MT | | | | | | |

TABLE 9
Effect of ACCELLERASE on antifoam consumption using pure
glucose as substrate, strain Corynebacterium glutamicum SP 10238.

| | 20% antifoam (ml) |
|---|---|
| Blank | 130 |
| Accellerase DUET @ | 100 |
| 4.5 kg/MT | |

The results showed that additional ACCELLERASE DUET during glutamic acid fermentation significantly decreased broth viscosity, slightly decreased residual glucose and slightly increased glutamic acid production, and at the same time reduced the foaming formation and decreased antifoam consumption 30%.

### Example 8

### Effect of Transglucosidase on Lactic acid production using pure sucrose as Substrate: Lactobacillus rhamnosus

Bacterial biomass collected from an agar plate transferred into seed culture medium in 300 mL flasks. Cultivate on a NBS rotary shaker at 150 rpm and 37° C., and the working volume 100 mL/300 mL flask, cultivate about 14˜18 hrs and maintain the OD above 0.5. The fermentation medium was sterilized at 121° C. for 12 min, cooled down the temperature to 40° C., the initial sucrose was 80 g/L (40 g/500 ml), controlled pH at 6.5 by addition of ammonium hydroxide, samples were withdrawn every three or four hours to measure sucrose and lactic acid, from 20 hrs to 32 hrs feed high concentration of sucrose 150 ml (100 g/150 m). TrTG was added at the beginning of the fermentation (0 h), 20 h, 21 h, with 0.145 g, 0.145 g, and 0.140 g separately. The total dosage of TrTG was 3.07 Kg/MT ds sucrose, analyzed Lactic acid production, broth viscosity after fermentation.

TABLE 10
Effect of TrTG on Lactic acid fermentation yield, residual sucrose
at the end of fermentation and viscosity of fermentation broth using
pure sucrose as substrate, strain Lactobacillus rhamnosus

| | Fermen- | | | | | Lactic | | |
|---|---|---|---|---|---|---|---|---|
| | tation | Total | Lactic | NH3•H2O | Final | acid | Residual | |
| | time | sucrose | acid | volume | volume | yield | sucrose | Viscosity |
| | (hrs) | (g) | (g/L) | (ml) | (ml) | (%) | (g/L) | (mpas) |
| Control | 49 | 140 | 20 | 10 | 660 | 14.29 | / | 28.21 |
| TrTG | 49 | 140 | 31 | 10 | 660 | 22.14 | / | 16.21 |
| @ 3.07 kg/MT | | | | | | | | |

The results showed that additional TrTG during lactic acid fermentation significantly decreased broth viscosity and increased lactic acid production.

### Example 9

### Effect of Transglucosidase on Lactic Acid Production Using Pure Glucose as Substrate: Lactobacillus rhamnosus

Bacterial biomass collected from an agar plate transferred into seed culture medium in 300 mL flasks. Cultivate on a NBS rotary shaker at 150 rpm and 37° C., and the working volume 100 mL/300 mL flask, cultivate about 14˜18 hrs and maintain the OD above 0.5. The fermentation medium was sterilized at 121° C. for 12 min, cooled down the temperature to 40° C., the initial glucose was 80 g/L (40 g/500 ml), controlled pH at 6.5 by addition of ammonium hydroxide, samples were withdrawn every three or four hours to measure sucrose and lactic acid, from 4 hrs to 22 hrs feed high concentration of glucose 150 ml (100 g/150 m). TrTG was added at the beginning of the fermentation (0 h), 10 h, 22 h, with 0.150 g, 0.150 g, and 0.300 g separately. The total dosage of TrTG was 4.28 Kg/MT ds glucose, analyzed Lactic acid production, residual sugar, broth viscosity after fermentation.

TABLE 11
effect of TrTG on Lactic acid fermentation yield, residual glucose
at the end of fermentation and viscosity of fermentation broth using
pure glucose as substrate, strain Lactobacillus rhamnosus

| | Fermen- | | | Diluted | | Lactic | | |
|---|---|---|---|---|---|---|---|---|
| | tation | Total | Lactic | NH3•H2O | Total | acid | Residual | |
| | time | glucose | acid | volume | volume | yield | glucose | Viscosity |
| | (hrs) | (g) | (g/L) | (ml) | (ml) | (%) | (g/L) | (mpas) |
| Control | 71 | 140 | 97.4 | 100 | 750 | 52.09 | 64.2 | 15.72 |
| TrTG | 71 | 140 | 103.6 | 230 | 880 | 65.16 | 42.3 | 12.97 |
| @ 4.28 kg/MT | | | | | | | | |

The results showed that additional TrTG during lactic acid fermentation significantly decreased broth viscosity, residual glucose and increased lactic acid production.

### Example 10

### Effect of OPTIMASH BG on Lactic Acid Production Using Pure Glucose as Substrate: Lactobacillus rhamnosus

Bacterial biomass collected from an agar plate transferred into seed culture medium in 300 mL flasks. Cultivate on a NBS rotary shaker at 150 rpm and 37° C., and the working volume 100 mL/300 mL flask, cultivate about 14˜18 hrs and maintain the OD above 0.5. The fermentation medium was sterilized at 121° C. for 12 min, cooled down the temperature to 45° C., the initial glucose was 80 g/L (40 g/500 ml), controlled pH at 6.5 by addition of ammonium hydroxide, samples were withdrawn every three or four hours to measure sucrose and lactic acid, from 6 hrs to 22 hrs feed high concentration of glucose 150 ml (100 g/150 m). OPTIMASH BG was added at the beginning of the fermentation (0 h), 22 h, with 0.150 g, 0.220 g separately. The total dosage of OPTIMASH BG was 3.70 Kg/MT ds glucose, analyzed Lactic acid production, residual sugar, broth viscosity after fermentation.

TABLE 12
effect of OPTIMASH BG on Lactic acid fermentation yield, residual glucose
at the end of fermentation and viscosity of fermentation broth using
pure glucose as substrate, strain Lactobacillus rhamnosus

| | Fermen- | | | Diluted | | Lactic | | |
|---|---|---|---|---|---|---|---|---|
| | tation | Total | LA | NH3•H2O | Total | acid | Residual | |
| | time | glucose | production | volume | volume | yield | glucose | Viscosity |
| | (hrs) | (g) | (g/L) | (ml) | (ml) | (%) | (g/L) | (mpas) |
| Blank | 46 | 140 | 69.8 | 150 | 800 | 55.84 | 56.9 | 8.10 |
| OPTIMASH BG | 46 | 140 | 108.3 | 190 | 840 | 77.36 | 21.5 | 0.00 |
| @3.7 Kg/MT | | | | | | | | |

The results showed that additional OPTIMASH BG during lactic acid fermentation significantly decreased broth viscosity, residual glucose and increased lactic acid production.

1. A method of making a fermentation product comprising;
fermenting a feedstock with a fermenting microorganism in a fermentation broth to make a fermentation product, wherein the fermenting comprises a transglycosidase, and wherein the fermentation product has a higher yield than a control fermentation lacking the transglycosidase.

2. The method of claim 1 further comprising recovering the fermentation product.

3. The method of claim 1 wherein the fermentation product is lysine, lactic acid, or glutamic acid.

4. The method of claim 1 wherein the fermenting organism is Brevibacterium flavum, Corynebacterium glutamicum 10178, Corynebacterium glutamicum 10238, or Lactobacillus rhamnosus.

5. The method of claim 1 wherein the transglycosidase comprises SEQ ID NO:1, or a polypeptide have at least 80%, at least 85%, at least 90%, at least 95%, at least 98%, or even at least 99% amino acid sequence identity to SEQ ID NO: 1.

6. The method of claim 1 wherein the transglycosidase is produced in a Trichoderma.

7. The method of claim 1 wherein the feedstock comprises starch liquifact, granular starch, pure glucose, pure sucrose, or sugar syrup.

8. The method of claim 1 further comprising foam reduction and/or viscosity reduction in the fermentation broth.

9. The method of claim 1 wherein the TrTG is present at 1-6 Kg/MT, 2-5 Kg/MT, or 2-4 Kg/MT.

10-12. (canceled)

13. A The method of claim 15, wherein
the fermenting comprises a TrTG Trichoderma transglucosidase, wherein the lysine has a higher yield compared to a control fermentation lacking the Trichoderma transglucosidase, and wherein the fermentation broth has reduced viscosity compared to a control fermentation lacking the Trichoderma transglucosidase.

14. A The method of claim 15, wherein
the fermenting comprises an Aspergillus niger transglucosidase, wherein the lysine has a higher yield compared to a control fermentation lacking the Aspergillus niger transglucosidase.

15. A method of making lysine comprising;
fermenting a feedstock with Brevibacterium flavum in a fermentation broth to make lysine, wherein the fermenting comprises a transglucosidase, wherein the lysine has a higher yield compared to a control fermentation lacking the transglucosidase; or
fermenting a feedstock with Brevibacterium flavum in a fermentation broth to make lysine, wherein the fermenting comprises a betaglucanase, xylanase and cellulase enzyme complex, wherein the lysine has a higher yield compared to a control fermentation lacking the betaglucanase, xylanase and cellulase enzyme complex.

16. The method according to claim 14, wherein the feedstock comprises pure glucose or sugar syrup.

17. (canceled)

18. The method according to claim 14 further comprising foam reduction and/or viscosity reduction.

19. A method of making glutamic acid comprising;
fermenting a feedstock with Corynebacterium glutamicum SP 10238 in a fermentation broth to make glutamic acid, wherein the fermenting comprises a TrTG, wherein the fermentation broth has reduced viscosity compared to a control fermentation lacking the TrTG, and wherein the fermentation broth has reduced foam compared to a control fermentation lacking the TrTG; or
fermenting a feedstock with Corynebacterium glutamicum SP 10238 in a fermentation broth to make glutamic acid, wherein the fermenting comprises an exoglucanase, a endoglucanase, a β-glucosidase, and a xylanase, wherein the fermentation broth has reduced viscosity compared to a control fermentation lacking the exoglucanase, the endoglucanase, the β-glucosidase, and the xylanase, and wherein the fermentation broth has reduced viscosity compared to a control fermentation lacking the exoglucanase, the endoglucanase, the β-glucosidase, and the xylanase.

20. (canceled)

21. A method of making lactic acid comprising;
fermenting a feedstock with Lactobacillus rhamnosus in a fermentation broth to make lactic acid, wherein the fermenting comprises a TrTG, wherein the lactic acid has a higher yield compared to a control fermentation lacking the TrTG, and wherein the fermentation broth has reduced viscosity compared to a control fermentation lacking the TrTG; or
fermenting a feedstock with Lactobacillus rhamnosus in a fermentation broth to make lactic acid, wherein the fermenting comprises a betaglucanase, xylanase and cellulase enzyme complex, wherein the lactic acid has a higher yield compared to a control fermentation lacking the betaglucanase, xylanase and cellulase enzyme complex, and wherein the fermentation broth has reduced viscosity compared to a control fermentation lacking the betaglucanase, xylanase and cellulase enzyme complex.

22. The method according to claim 21, wherein the feedstock comprises pure glucose or sugar syrup.

23-35. (canceled)

36. The method according claim 15, wherein the feedstock comprises pure glucose or sugar syrup.

37. The method according to claim 15, further comprising foam reduction and/or viscosity reduction.

38. The method according to claim 17, further comprising foam reduction and/or viscosity reduction.
